(19) United States
(12) Patent Application Publication
(10) Pub. No.: US 2026/0151024 A1
(43) Pub. Date: Jun. 4, 2026

(54) FLEXIBLE, THIN-FILM MINIATURIZED ENDOSCOPE AND METHOD OF USE THEREOF

(71) Applicant: CARNEGIE MELLON UNIVERSITY, Pittsburgh, PA (US)

(72) Inventors: Maysam Chamanzar, Pittsburgh, PA (US); Mohammad Hassan Malekoshoaraie, Pittsburgh, PA (US); Jay Reddy, Pittsburgh, PA (US); Vishal Jain, Pittsburgh, PA (US)

(73) Assignee: CARNEGIE MELLON UNIVERSITY, Pittsburgh, PA (US)

(21) Appl. No.: 19/455,124

(22) Filed: Jan. 21, 2026

Related U.S. Application Data
(63) Continuation-in-part of application No. 17/486,351, filed on Sep. 27, 2021, now Pat. No. 12,343,554.
(60) Provisional application No. 63/084,133, filed on Sep. 28, 2020.
(60) Provisional application No. 63/801,446, filed on May 7, 2025.

(51) Int. Cl. A61B 1/04; A61B 1/00; A61B 1/01; A61B 1/05; A61B 1/06; A61B 1/07

(52) U.S. Cl. CPC A61B 1/043; A61B 1/00045; A61B 1/00112; A61B 1/01; A61B 1/05; A61B 1/0625; A61B 1/07

(57) ABSTRACT

Disclosed herein is an implantable or wearable, flexible, miniaturized microimaging suited for localized brain fluorescent imaging. The microimaging device is implemented on a Parylene photonics platform, which is biocompatible and fabricated using scalable planar microfabrication techniques, enabling customizable design, size, and number of channels (pixels). Operating across the entire visible spectrum. The microimaging device functions effectively both for coherent and scattered light. The microimaging device can be implanted into the brain of freely moving animals, where one or more waveguides could be dedicated to light delivery and the rest of the waveguides for collecting and relaying the fluorescent image to image the tissue structure and function.

### RELATED APPLICATIONS

This application claims the benefit of U.S. Provisional Ser. No. 63/801,446 , filed May 7, 2025, the contents of which are hereby incorporated herein by reference in their entirety.

This application is also a continuation-in-part of U.S. patent application Ser. No. 17/486,351, filed Sep. 27, 2021, which claims the benefit of U.S. Provisional Pat. App. 63/084,133, filed Sep. 28, 2020, the contents of which are incorporated herein by reference in their entireties.

### GOVERNMENT INTEREST

This invention was made with the support of the United States Government under contract 1926804 awarded by the National Science Foundation. The U.S. Government has certain rights in this invention.

### BACKGROUND

Optical biomedical imaging is a versatile and powerful technique that employs ultraviolet, visible, or near-infrared light to capture images of biological samples based on light-matter interaction through reflection, refraction, absorption, scattering, or fluorescent emission. It provides detailed structural, functional, and molecular information. Over the past few decades, it has been widely used in basic science research as well as clinical diagnostics.

Several optical imaging modalities have been implemented. For example, fluorescent imaging has been used for molecular imaging, providing helpful insights into biological processes. This technique involves illuminating the tissue with light at a specific wavelength to excite fluorophores, which are molecules that fluoresce upon excitation and are either genetically expressed or chemically attached to specific target cells. The excited fluorophores emit light at a longer wavelength, which is captured and filtered by the optical imaging system to provide detailed information about the location and function of the labeled cells. Fluorescent imaging is used in many different applications, such as imaging malignant cells, which are often difficult to distinguish from healthy cells using conventional white-light optical imaging. It has also played a key role in basic science discovery, especially in neuroscience, where it helps with monitoring neural tissue activity and identifying the role of specific cell types across populations of neurons to unravel the complex brain function that mediates behavior. In clinical applications, fluorescence imaging in guided surgery helps with the identification of tumor locations and boundaries during operations. Visualization of tumors during surgery enhances resection precision by minimizing harm to healthy tissue.

Single-photon, multiphoton, confocal microscopy, and optical coherence tomography (OCT) are commonly used biomedical optical imaging methods. The basic structure of an optical microscope consists of a lens system that projects the backscattered light from within the tissue onto an army of photodetectors to form the image of the underlying structure. However, traditional microscopes are typically limited to imaging superficial layers of tissue due to light scattering and attenuation, restricting the imaging depth and resolution. They are often bulky and have a limited field of view (FOV).

To address the bulkiness issue, miniaturized microscopes, called miniscopes, have been developed, which can be implanted into the tissue and offer enhanced maneuverability. The miniscopes are also composed of a compact lens system and a photodetector array (e.g., an image sensor), similar to conventional microscopes but in a much smaller form factor. They are primarily used in research, particularly in neuroscience, for imaging small, localized areas to monitor neural activity in live animal subjects using calcium imaging.

The invasiveness of implantable microscopy and endoscopy techniques depends on the accessibility of the target region of interest. When imaging a superficial layer of tissue such as skin, the technique is considered noninvasive. However, accessing deeper regions may require surgical removal of superficial tissue layers, rendering this method invasive.

Optical imaging of deeper organs due to light absorption and scattering in biological tissue requires endoscopic tools, including a long, rigid, or semi-flexible insertion tube that contains the light source and a miniaturized imaging system. Such systems are used for diagnostic and therapeutic purposes and are introduced through natural body orifices or small surgical openings in superficial tissues. Endoscopes are widely used in applications such as gastroenterology, cardiology, urology, pulmonology, endovascular interventions and orthopedics. Compared to microscopes, endoscopes offer greater compactness and maneuverability, resulting in a larger FOV. Endoscopes generally provide lower resolution than microscopes because they prioritize compactness, limiting the integration of high-resolution optics and sensors. Additionally, insertion of endoscopes causes tissue damage. While such damage may be less critical in surgical procedures, minimizing it is essential during diagnostic procedures to preserve tissue integrity and function. The state-of-the-art endoscopes are still relatively bulky and can severely damage the tissue.

There are different designs of endoscopes. An imaging system at the distal end equipped with a photodetector array chip, capturing images directly at the tip, is referred to as a distal-chip endoscope. Recent innovations in implantable imaging have introduced lensless devices capable of delivering light and collecting images directly on-chip. In contrast, in fiber-optic endoscopes, a fiber bundle relays the image from the distal end to a photodetector array chip at the proximal backend. Each individual fiber relays a separate pixel of the scene to the image sensor array. The distal-chip endoscope has higher image quality, whereas the fiber-optic endoscope, due to the limited density of optic fibers, has lower resolution but is simpler and more compact at the distal end, making it less invasive and more flexible.

A recent advancement in fluorescent imaging for neuroscience research related to fiber optic endoscopy is fiber photometry, which typically employs a single fiber that penetrates deep into the brain to detect light emitted from fluorescently tagged cells. This method produces a single-pixel image, offering limited information about specific cell types within a small volume near the fiber tip, without revealing more details. Increasing the number of pixels is desired, but using a bundle of fibers can become prohibitively invasive.

Despite ongoing efforts to miniaturize endoscopic tools, they often remain bulky, resulting in significant tissue damage. Using fiber bundles has limitations, including the relatively large diameter of fiber bundles, rigidity, and limited FOV, especially because all of the fibers terminate at the same distal plane. Additionally, the tightly packed honeycomb arrangement of the fiber strands limits the customizability of the pixel layout.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is the use of miniaturized flexible thin-film integrated photonic waveguides as the basis of next generation, ultra-compact endoscopes which address the limitations of fiber bundle endoscopes by reducing the mechanical mismatch with soft tissue and offering a customizable design of the imager. By leveraging planar microfabrication processes, a high density of on-chip optical waveguides can be implemented on flexible polymer substrates. These thin-film endoscopes enable seamless integration with robotic surgical tools via lamination, and their customizable pixel arrangements support targeted illumination and imaging.

Also disclosed herein is a miniaturized, thin-film flexible endoscope designed for localized optical imaging (i.e., a “microimaging device”). The microimaging device utilizes a dense array of polymer photonic waveguides forming a fully flexible, compact, and biocompatible waveguide array fabricated using a scalable planar microfabrication process. Unlike traditional fiber bundles, the microimaging device can be seamlessly integrated with light sources, detectors, and surgical tools, simplifying system design while its thin profile minimizes tissue damage for deep tissue penetration. Furthermore, the microimager has a customizable pixel arrangement that facilitates precise, localized illumination and imaging, and the device can be incorporated into existing surgical tools to reduce procedural complexity.

### BRIEF DESCRIPTION OF THE DRAWINGS

By way of example, specific exemplary embodiments of the disclosed system and method will now be described, with reference to the accompanying drawings, in which:

FIG. 1 is a schematic rendering of an imaging system using the microimaging device disclosed herein.

FIG. 2 shows various illustrations showing details of the design of the microimaging device.

FIG. 3 shows the steps of the microfabrication of the microimaging device.

FIGS. 4A-D show optical microscope images of fabricated device.

### DEFINITIONS

As used herein, the terms “approximate” or “approximately” should be interpreted as meaning a value within±10% of a stated value.

### DETAILED DESCRIPTION

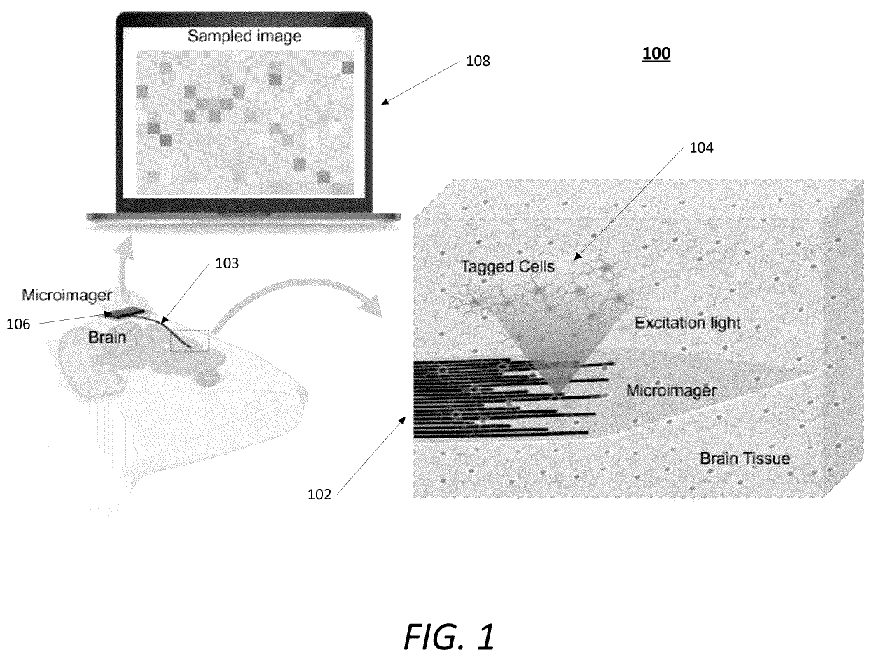

FIG. 1 is a schematic illustration of a system using the microimaging device. The microimaging device and system is explained in the context of a fluorescent imaging application, however, as would be realized by one of skill in the art, the system is not limited to that specific application and may be used with other optical modalities.

For fluorescent imaging, the microimager is capable of delivering excitation light and collecting the fluorescent emission from within tissue. The microimaging device is composed of an array of flexible optical waveguides 102 along a thin-film shank 103. In the case of an implantable device, the thin-film material should be biocompatible. The waveguides 102 relay pixelized fluorescent images (for example, of the fluorescent protein-tagged neurons 104 within the brain) collected at a distal end of the microimager to an image sensor located at a proximal end of the microimager and housed in the backend optoelectronics 106. The collected image can be displayed on display 108.

The waveguides 102 are bidirectional (i.e., they can both collect and deliver light). In the system shown in FIG. 1, some of the waveguides in the array can be designated for light delivery, while the remaining waveguides (preferably grouped together such as to create an image at the image sensor) can be used to collect the fluorescent emission. This way, an ultracompact microimaging device can be implemented with embedded light delivery and light collection waveguides and an integrated image sensor array, both of which can be coupled to the waveguides at an integrated platform located at a proximal end of the device, outside of the subject.

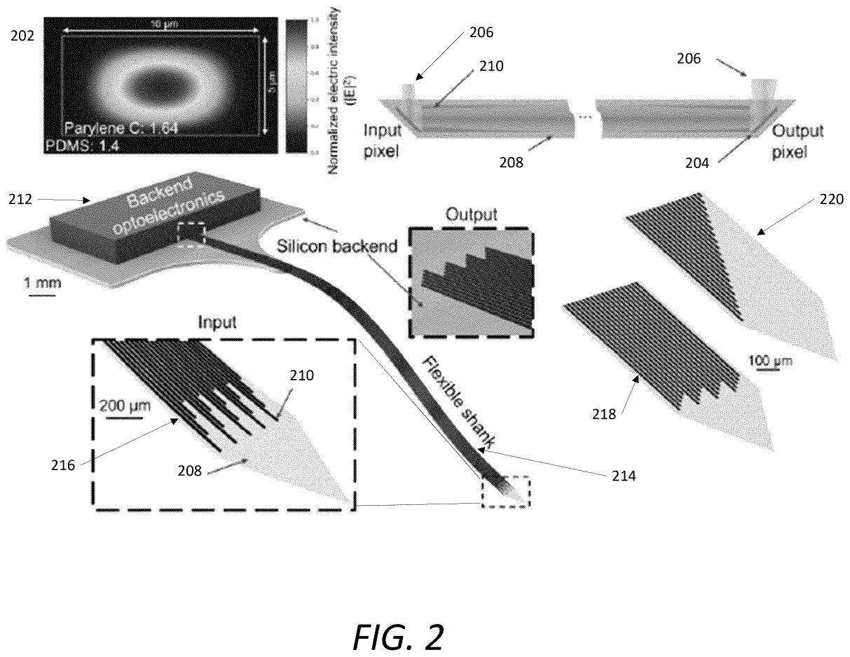

To implement the microimager, a biocompatible, flexible waveguide platform capable of efficiently transferring light is required. In preferred embodiments, Parylene photonics is used as the flexible integrated photonics platform. In preferred embodiments, the waveguide cores are composed of Parylene-C, a polymer with a high refractive index (n=1.639) that remains transparent across the visible optical spectrum. Polydimethylsiloxane (PDMS) is used for cladding surrounding each waveguide core, due to its lower refractive index (n=1.4) compared to Parylene-C. This combination of Parylene-C and PDMS provides a significant index contrast (Δn=0.239) among biocompatible polymers, effectively confining the optical mode to the waveguide cores, as visualized in FIGS. 2, 202 . The waveguide platform can be realized using any two materials that have different refractive indices, including various types of polymers.

FIG. 2 Illustrates various design details of the microimaging device. The Parylene photonic platform is realized through a scalable micromachining process on the polymer layers sitting on a silicon substrate. The flexible polymers are subsequently released from the substrate to realize a fully flexible implantable or wearable device 214. FIG. 2 shows the flexible substrate 208 and one waveguide 210, with one end being defined as an input pixel and the opposite end being defined as the output pixel.

A key feature of the Parylene photonics platform is the inclusion of embedded micromirror structures 204 disposed at each end of each waveguide, which facilitate broadband input/output light coupling and imaging. The micromirrors 204 are preferably disposed at an approximate 45° angle with respect to a longitudinal axis of the device, such as to enable approximate 90° bidirectional out-of-plane light coupling 206 and imaging. Unlike traditional optical waveguides and fibers that rely on end-firing light emission from the end facet, this out-of-plane approach is preferred for achieving high spatial resolution imaging and illumination along the thin-film shank. Additionally, it allows for the integration of backend optoelectronics on the surface, resulting in a compact design suitable for chronic experiments on freely moving subjects. Image sensors, located as part of the backend optoelectronics 212 can be directly integrated onto the surface of the substrate 208 for light detection. In preferred embodiments, charge-coupled devices (CCD) or complementary metal-oxide-semiconductor (CMOS) sensors are used as the image sensors and are coupled to a subset of the total number of defined waveguides.

Furthermore, light sources can be coupled from the surface using an optical fiber or by integrating a laser diode located on backend platform 212 with the optical waveguides. For the microimaging device disclosed herein, both the light source(s) and image sensor are coupled to separate subsets of the waveguides at the proximal end of the device in the backend electronics module 212, which also houses supporting electronics for the one or more light sources and the image sensor. The distal end of the device, which is typically implanted in a subject, is thus used to both deliver illumination light through one set of waveguides and to collect images through a second set of waveguides. It should be noted that the illumination delivered by the device can be for purposes of obtaining imagery but may also be for therapeutic purposes.

The width of the optical waveguides can vary depending on the fabrication constraints, microimaging resolution, and the number of waveguides on the flexible shank 214. In terms of fabrication, optical photolithography is used to pattern different device layers with a resolution of 1 μm. Therefore, theoretically, the smallest waveguide width and pixel size is 1 μm. However, the optical propagation loss of waveguides smaller than 5 μm is very high. To achieve smaller waveguides, the fabrication process must be optimized by using high-resolution lithography techniques, such as electron beam lithography, and optimizing the etching processes.

In exemplary fabricated devices the cross-section of the Parylene waveguide core is 10 μm (width)×5 μm (thickness) with a pitch size of 20 μm. The dense waveguide array consists of 20 waveguides, making the width of flexible shank 214 400 μm. In preferred embodiments, the cladding thickness is 1 μm, as this thickness is sufficient for the waveguide to effectively confine the optical mode. Therefore, the overall thickness of the implantable/wearable flexible shank 214 is 7 μm, resulting in a thin device that minimizes damage to the surrounding tissue wen implanted.

The implantable/wearable flexible microimaging device can be of varying lengths, ranging from 4 to 8 mm, depending on the target depth of interest. The waveguide portion on the silicon (Si) backend is 2 mm, resulting in an overall optical waveguide length of 6 to 10 mm. During the microfabrication process, to release the flexible polymer device from the Si substrate, a small portion of rigid Si is retained at the proximal end of the microimager to facilitate integration with the optoelectronics system 212 and to ease handling during assembly. Each waveguide relays optical intensity from the input port to the output port, forming a “pixel” of the illumination or imaging portion of the microimaging device.

One embodiment of the microimager has 20 pixels at the input/output ports, arranged in a 2D periodic step-shaped arrangement consisting of 5 rows of 4 pixels, as shown by reference number 216 in FIG. 2. This design achieves a compact layout while ensuring that the pixels are separated to minimize interference between adjacent pixels, facilitating integration with an image sensor or a photodetector array. In general, the input pixel locations and the number of pixels can be customized in any arbitrary 2D arrangement on the shank 214 to provide the desired coverage or resolution within the imaging area of interest. For example, for imaging a larger area, a sparse 2D arrangement might be desired, and for imaging a local region with higher resolution, a dense arrangement is recommended. FIGS. 2, 216 shows an example of a grid-like 2D arrangement of input pixels, and FIGS. 2, 218, 220 show additional 2D designs with different linear pixel arrangements.

Parylene-C, used as the waveguide 210 core material in the microimager, exhibits low autofluorescence in the green and red wavelengths of the visible light spectrum while showing higher autofluorescence under UV and blue excitation due to photo-induced dehydrogenation and oxidation. Although the excitation light propagates through the Parylene waveguides, autofluorescence is minimized by using appropriate emission filters before the camera and selecting GFP fluorophores with emissions outside high-autofluorescence wavelengths. Regarding power handling, the optical power used is well below the damage threshold for Parylene-C, and no photothermal degradation is observed during operation.

To implement the microimaging device, a Parylene photonics platform is fabricated. Parylene photonics is fabricated at the wafer scale using a planar micromachining process to form the waveguide structures. This process is disclosed in U.S. Pat. No. 12,343,554, however, several improvements to the microfabrication process of the referenced patent are disclosed herein.

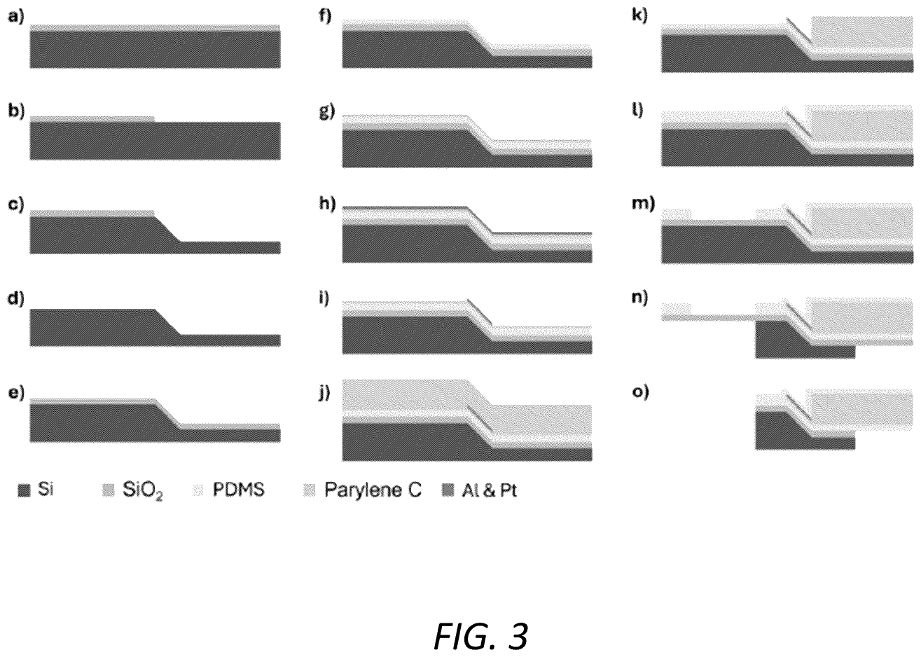

FIG. 3 shows the steps of the improved microfabrication process. The crystalline planes of the wafer are important for the Parylene photonics platform to form the approximate 45° sidewalls in a Si mold that define the surface of the micromirror structures 204 and shape of the waveguide. The fabrication process begins with a 4-inch Si wafer (n{100}) that had a 1 μm layer of thermal oxide (SiO2) grown on top (FIG. 3a). The mask design features were oriented at 45° with respect to the (100) plane, which is necessary to expose the (110) crystal plane and define the 45° Si sidewalls that form the micromirror surface. The oxide layer was patterned using the described mask through optical lithography and anisotropic reactive ion etching (RIE), which then served as the hard mask for etching the Si (FIG. 3b). Using the oxide hard mask, the waveguide template was etched in silicon in a solution of 2 M potassium hydroxide (KOH) mixed with 60 ppm Triton X-100 surfactant to achieve a smooth mold with angular sidewalls by revealing the (110) plane and reaching a target depth of approximately 60 μm (FIG. 3c). The oxide hardmask is then removed in 49% Hydrofluoric acid (FIG. 3d). A300-nm SiO2 layer is deposited conformally on the patterned Si surface using plasma-enhanced chemical vapor deposition (PECVD) as a sacrificial layer to enable the device release from the Si mold (FIG. 3e).

To form the substrate of the microimager and lower cladding for the waveguide structure, PDMS is used. Due to its high viscosity, spin-coating thin conformal layers of PDMS is challenging. Therefore, it is first diluted in hexane with a ratio of 1:10, and spin-coated (60 sec@2000 rpm) onto the Si mold. The film is degassed (1 mTorr) and oven-baked (1 hr@100°C.) to cure (FIG. 3f).

The micromirrors 204 need to be patterned on 45° sidewalls of the Si mold. Because of the low surface energy of PDMS, a very thin (300 nm) layer of Parylene-C film is deposited on the PDMS using chemical vapor deposition (CVD) to act as an adhesion layer for the photoresist and enable optical lithography (FIG. 3g). Metal layers are then deposited by evaporation of 5 nm Pt and 100 nm Al (FIG. 3h), and the micromirrors 204 are patterned using a lift-off process through acetone soaking followed by sonication (FIG. 3i). Pt serves as a strong adhesion layer to Parylene, while Al is an effective mirror surface due to its high reflectance across the visible spectrum.

The waveguide core is then fabricated in Parylene-C. A 5 μm layer of Parylene-C was deposited using CVD on top of the thin adhesion layer previously deposited during the micromirror fabrication step (FIG. 3j), To define the outline of the individual waveguide cores and remove unwanted Parylene-C regions, a thick photoresist (12 μm) etch mask is used. The waveguide patterns are aligned to the micromirrors 204 using optical lithography, The patterns are subsequently transferred to Parylene-C through anisotropic oxygen (02) plasma etching using RIE. PDMS acted as an etch-stop layer because this polymer is resistant to etching by 02 plasma alone. Finally, the photoresist etch mask was removed using acetone (FIG. 3k).

A 1 μm layer of diluted PDMS is then spin-coated as the upper cladding on Parylene-C core waveguides, similar to the process used for the lower cladding (FIG. 3l). Next, the outline of the microimager is defined using the thick photoresist etch mask. Then, the entire 2 μm PDMS claddings at the device outline are etched using O2 and sulfur hexafluoride plasma in an RIE process to singulate the individual devices (FIG. 3m).

To release fully flexible devices, the Si backend of the microimager is retained while the excess silicon is removed. This is achieved by patterning the backside of the Si substrate using a thick photoresist etch mask, followed by a deep reactive ion (DRIE) etching process, such as the Bosch process. The etching process was performed in two steps: a 12-second etch with 02 and SF 6 plasma, followed by an 8-second passivation with C4F8. The sacrificial oxide layer serves as the etch stop to protect the backside of flexible shank (FIG. 3n). After the Si is dissolved, the etch mask is removed in acetone and the sacrificial layer is stripped using buffered HF acid, leading to a released, flexible device with a Si backend (FIG. 3o). The devices are thoroughly rinsed in deionized water after release to avoid contamination of biological tissues by the process chemicals. The ability to keep the Si backend while achieving a fully flexible shank 214 represents a significant advancement, as it made handling easy during assembly and facilitates integration with the backend optoelectronics, thereby enhancing their performance and versatility in neural interfaces.

In a final step, laser fibers of other light sources (e.g., an integrated laser diode) can be coupled to the proximal ends of the subset of waveguides being used for illumination, and an image sensor can be coupled to the proximal ends of the subset of waveguides being used for imaging.

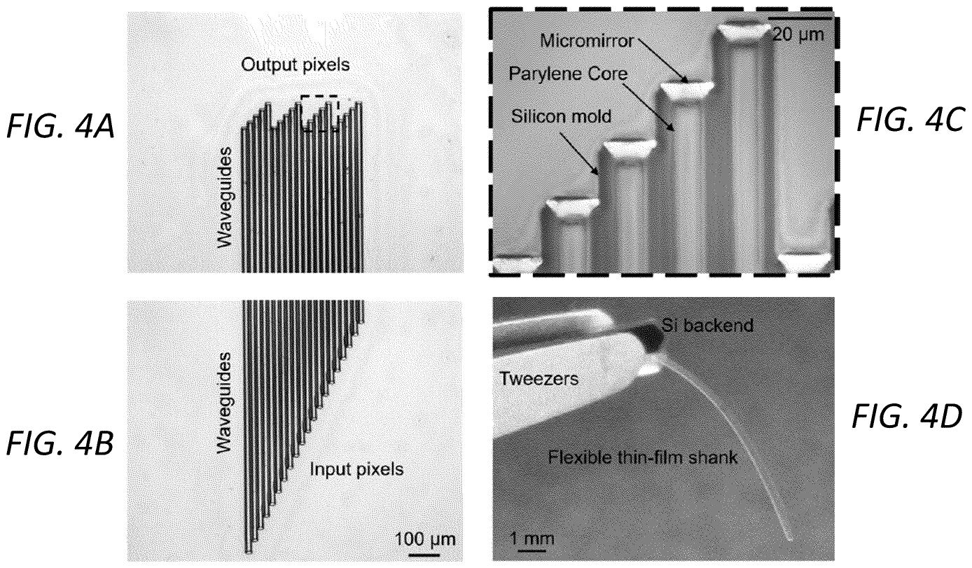

FIGS. 4A-D show optical microscope images of fabricated device. FIG. 4A shows a waveguide array and output pixels on the fabricated on-chip microimager. The inset of FIG. 4C provides a closer view of the output pixels, highlighting different components of the microimaging device. FIG. 4B shows the waveguide array and input pixels. FIG. 4D shows the flexible released device with the silicon backend.

As would be realized by one of skill in the art, many variations on the implementations and processes discussed herein which fall within the scope of the invention are possible. Specifically, many variations on the parameters used in the steps of the fabrication process are possible. Additionally, many variations of the arrangement of the input and output ports of the waveguides, as well as the number of the waveguides are all intended to be within the scope of the invention. The invention is not meant to be limited to the particular exemplary embodiments disclosed herein. Moreover, it is to be understood that the features of the various embodiments described herein were not mutually exclusive and can exist in various combinations and permutations, even if such combinations or permutations were not made express herein, without departing from the spirit and scope of the invention. Accordingly, devices disclosed herein are not to be taken as limitations on the invention but as an illustration thereof.

1. A method comprising:
inserting a distal end of a microimaging device into the body of a subject;
wherein the microimaging device comprises:
a flexible body having multiple waveguides defined therein, each waveguide having a coupling structure defined at each end thereof to redirect travel of light in an out-of-plane direction with respect to the flexible body;
delivering light from one or more light sources via the coupling structures on proximal ends of a first subset of the waveguides such as to illuminate an internal structure of the
body of the subject by light delivered via the coupling structures on distal ends of the first subset of waveguides; and
capturing an image of the internal structure via the coupling structures on proximal ends of a second subset of the waveguides, the image collected via the coupling structures on distal ends of the second set of waveguides.

2. The method of claim 1 wherein the light delivered from the one or more light sources is ultraviolet, visible or near-infrared.

3. The method of claim 1 wherein the image is collected via reflection, refraction, absorption, scattering or fluorescent emission of the internal structure.

4. The method of claim 1 wherein the microimaging device is used for fluorescent imaging.

5. The method of claim 1 wherein the one or more light sources are integrated laser diodes.

6. A device comprising:
a flexible substrate having one or more trenches defined therein;
one or more optical waveguides formed in the one or more trenches;
a pair of out-of-plane input and output coupling structures integrally formed in the flexible substrate at opposite ends of each waveguide to redirect travel of light in each of the waveguides to an out-of-plane direction with respect to the flexible substrate;
one or more light sources coupled to a proximal end of a first subset of the waveguides via the coupling structures; and
an image sensor coupled to a proximal end of a second subset of the waveguides via the coupling structures;
wherein a structure illuminated via the first subset of waveguides is imaged via the second subset of waveguides.

7. The device of claim 6 wherein the flexible substrate is laminated to a surgical instrument.

8. The device of claim 6 wherein the flexible substrate is composed of silicon.

9. The device of claim 6 wherein the one or more waveguides are composed of Parylene-C.

10. The device of claim 6 wherein the one or more waveguides are surrounded by cladding to confine the optical mode within the waveguides.

11. The device of claim 10 wherein the refractive index of the cladding material is lower than the refractive index of the waveguide material.

12. The device of claim 11 wherein the waveguides are composed of Parylene-C and the cladding is composed of PDMS.

13. The device of claim 6 wherein the input and output coupling structures are micromirrors.

14. The device of claim 13 wherein the micromirrors are angled at an approximate 45° angle with respect to a longitudinal axis of the flexible substrate.

15. The method of claim 6 wherein the one or more light sources are integrated laser diodes.

16. The device of claim 6 further comprising:
a backend platform attached to or integral with the flexible body wherein the one or more lights sources and the image sensor are housed and coupled to the waveguides; and
electronic components supporting the one or more lights sources and the image sensor.

17. A system comprising:
the device of claim 16; and
a display device;
wherein the image sensor outputs an image for display on the display device.
